(19) United States
(12) Patent Application Publication
(10) Pub. No.: US 2014/0099656 A1
(43) Pub. Date: Apr. 10, 2014

(54) RAPID DETECTION OF HISTAMINE IN FOOD AND BEVERAGES

(71) Applicant: Joseph F. Krebs, Austin, TX (US)

(72) Inventor: Joseph F. Krebs, Austin, TX (US)

(73) Assignee: BIOO SCIENTIFIC CORPORATION, Austin, TX (US)

(21) Appl. No.: 14/043,488

(22) Filed: Oct. 1, 2013

Related U.S. Application Data
(60) Provisional application No. 61/708,134, filed on Oct. 1, 2012.

(51) Int. Cl. C12Q 1/32

(52) U.S. Cl. CPC C12Q 1/32
USPC 435/26; 435/309.1

(57) ABSTRACT

Described herein is a rapid, semi-quantitative sampling device which may rapidly detect histamine in food, beverage and feed samples. The device uses an enzyme (impregnated in a sampling pad) to specifically convert histamine present in samples to a visual colorimetric signal. The color change of the pad is proportional to the amount of histamine present in the sample.

### PRIORITY CLAIM

This application claims the benefit of U.S. Provisional Application No. 61/708,134 filed on Oct. 1, 2012.

### BACKGROUND OF THE INVENTION

1. Field of the Invention

The present invention generally relates to the detection of histamine in food, feed and beverage samples.

2. Description of the Relevant Art

Scombroid (histamine) poisoning is the second leading cause of food-borne illness in the United States, accounting for nearly one in fifty of the total cases of food-borne illness in the U.S. according to the most recent CDC reports (CDC website, 2007). Scombroid poisoning is caused by histamine-producing bacteria such as V. alginolyticus (Vibrio), R. planticola and R. ornithinolytica which can grow on improperly handled fish. When allowed to grow at elevated temperatures, these bacteria convert histidine into histamine which accumulates in the meat over time. Ingestion of the histamine contaminated fish (50-2,000 ppm) triggers an acute allergic response in humans which can lead to nausea, vomiting, diarrhea, hypotension, rashes and hives.

Scombroid (histamine) poisoning can be prevented by testing the amount of histamine in seafood to identify contaminated lots. Aside from proper handling techniques, the key to eliminating scombroid poisoning is to identify histamine-contaminated fish samples before they enter the public food supply. Histamine contamination can also occur in other food and beverage types, such as cheese, wine and milk, as well. Therefore, histamine testing can be widely employed throughout the food supply to prevent accidental ingestion of contaminated food and beverages.

Current histamine detection methods include high-performance liquid chromatography, mass spectrometry and ELISA (the low molecular weight of histamine limits its antigenicity, so these ELISA kits usually include a chemical modification step to allow antibody recognition). Most current quantitative methods for histamine determination require the use of lab equipment and scientific instrumentation. While several antibody-based lateral flow devices have been developed for field-testing, these devices provide a strictly qualitative result. Also, a given lateral flow device must be dedicated for the detection of histamine at or above a specific concentration and cannot be used for histamine screening in markets with different action limits. Furthermore, these lateral flow strip tests cannot provide a quantitative determination of histamine in samples. Collectively, the inherent limitations of the currently available analytical techniques impede their capacity to allow comprehensive histamine screening of seafood: currently only 2% of the supply of “scombroid-susceptible” fish is tested for histamine in the U.S.

The relatively expensive, time-consuming and labor-intensive nature of the currently-available detection methods for histamine dramatically limits their capacity to test large numbers of samples. This hampers the ability of analysts to test the overall food supply and quarantine contaminated samples. New, rapid testing methods are sorely needed. It would be highly beneficial to develop a simple, rapid method to detect histamine levels in food and beverage samples in the field as well as in the kitchen or retail environment.

### SUMMARY OF THE INVENTION

Histamine dehydrogenase (HDH) enzyme may be used in a sampling device that allows the rapid detection of histamine in food, feed and beverage samples. The sampling device includes a sampling pad made from an absorbent material (e.g., a cellulose fiber pad) coupled to the end of a solid support. The sampling pad is impregnated with histamine dehydrogenase enzyme. The sampling device is then inserted into a mixture of a sample mixed with a buffer and a coloring composition that includes a tetrazolium salt to put the pad in contact with the sample and reagents. The sampling device is then removed from the mixture and the device is incubated at room temperature to allow the reaction to proceed to completion. If histamine is present in the sample, the sampling pad will become colored; the sampling pad will remain colorless if there is no histamine in the sample.

In one embodiment, a method of detecting histamine in a sample, includes obtaining a liquid sample containing histamine from the sample, wherein the liquid sample is buffered at a pH of between about 8.5 to about 10; adding a coloring composition comprising a tetrazolium salt to the liquid sample to create a test sample; placing a sampling device into the test sample; removing the sampling device from the test sample; allowing a sampling pad of the sampling device to incubate for a time of less than five minutes, wherein a change in color of the sampling pad indicates the presence of histamine in the sample.

In an embodiment, a sampling device includes: a sampling pad impregnated with histamine dehydrogenase enzyme; and a support substrate, wherein the sampling pad is coupled to the support substrate; wherein the pad of the sampling device is placed into the test sample during use.

In an embodiment, a test kit for the visual detection of histamine in a sample includes: a sampling device as described herein; a coloring composition comprising a tetrazolium salt; and a buffer composition having a pH of between about 8.5 to about 10.

### BRIEF DESCRIPTION OF THE DRAWINGS

Advantages of the present invention will become apparent to those skilled in the art with the benefit of the following detailed description of embodiments and upon reference to the accompanying drawings in which:

FIG. 1 depicts a schematic drawing of a sampling device in the absence and presence of histamine.

While the invention may be susceptible to various modifications and alternative forms, specific embodiments thereof are shown by way of example in the drawings and will herein be described in detail. The drawings may not be to scale. It should be understood, however, that the drawings and detailed description thereto are not intended to limit the invention to the particular form disclosed, but to the contrary, the intention is to cover all modifications, equivalents, and alternatives falling within the spirit and scope of the present invention as defined by the appended claims.

### DETAILED DESCRIPTION OF THE PREFERRED EMBODIMENTS

It is to be understood the present invention is not limited to particular devices or methods, which may, of course, vary. It is also to be understood that the terminology used herein is for the purpose of describing particular embodiments only, and is not intended to be limiting. As used in this specification and the appended claims, the singular forms “a”, “an”, and “the” include singular and plural referents unless the content clearly dictates otherwise. Furthermore, the word “may” is used throughout this application in a permissive sense (i.e., having the potential to, being able to), not in a mandatory sense (i.e., must). The term “include,” and derivations thereof, mean “including, but not limited to.” The term “coupled” means directly or indirectly connected.

Histamine can be detected in food samples using a colorimetric enzymatic reaction. One potential enzymatic scheme employs the use of a histamine dehydrogenase (HDH) enzyme. This commercially available enzyme removes the histamine amino group via an oxidative deamination process to specifically form imidazole acetaldehyde (IAA): A detection scheme using HDH is set forth below:

Step 1: Histamine+H₂O+HDH_{oxidized}→IAA+NH₃+HDH_{reduced}

Step 2: HDH_{reduced}+mPMS(oxidized)→HDH_{oxidized}+mPMS(reduced)

Step 3: mPMS(reduced)+WST-8→mPMS(reduced)+formazan (colored)

The histamine dehydrogenase reaction (Step 1) can be monitored by coupling the subsequent oxidation of the reduced enzyme (spectrophotometrically) to reduce a tetrazolium salt (WST-8) to a colored formazan product (Step 3). In published reports, this reduction is mediated through an electron carrier such as 1-methoxyphenazine methosulfate (mPMS) which can readily transfer electrons from the enzyme to the tetrazolium (Step 2). The amount of histamine in the sample is proportional to the resulting amount of formazan produced as measured by the color change in the reaction. This reaction occurs quite rapidly in solution.

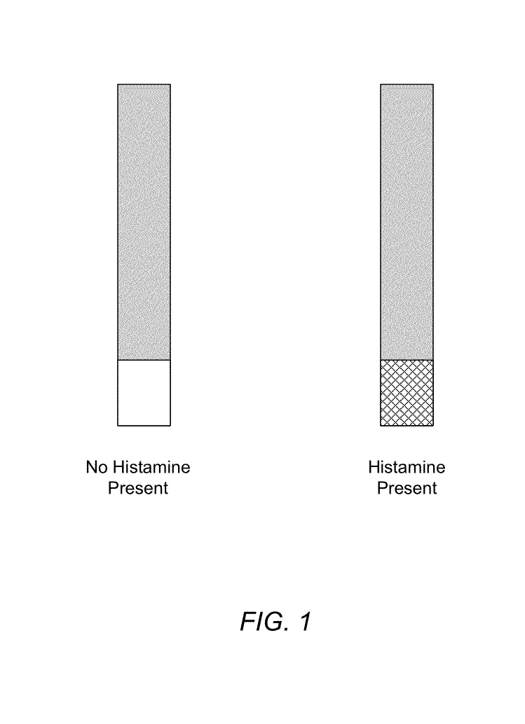

In an embodiment, a novel sampling device is described for the determination of histamine in food, beverage and feed samples which includes a sampling pad formed from an absorbent material coupled to a support substrate. The device relies on a colorimetric enzyme reaction which changes the color of a sampling pad of the device when the device comes into contact with liquid samples containing histamine. The sampling pad (e.g., a cellulose fiber pad) is attached to the end of the support substrate (e.g., a polymeric strip) which provides a way to manipulate the sampling pad without contacting the pad (see FIG. 1).

The sampling pad is impregnated with histamine dehydrogenase (HDH) enzyme by briefly soaking the sampling pad in a histamine dehydrogenase enzyme composition and allowing the pad to dry. The drying period allows the enzyme to bind into the pad in a stable form. The enzyme composition may contain a buffer (pH 9-9.5) to maintain the enzyme in active form. The enzyme composition may also contain trehalose to enhance the stability of the enzyme.

During testing a sample is collected from the food or beverage being tested. Samples may be collected directly from liquid containing foods or beverages. Solid or dry food or feed may be soaked in water to obtain a liquid or suspended sample for testing. The obtained sample may be treated with an aqueous acidic composition (e.g., 0.05% acetic acid) to form an acidic mixture. If a solid sample is used, any undissolved solids may be removed (e.g., by filtering or centrifuge) to form a clarified acidic mixture. If the original sample was liquid, the acidified liquid sample may be used without any further treatment. The acidified sample may be added to a neutralization buffer to form a liquid sample. The neutralization buffer may provide a liquid sample having a pH of between about 8.5 to about 10. In an embodiment, the buffer is a 0.6 M glycine buffer having a pH between about 8.5 to about 10, preferably between about 9 to about 9.5. A coloring composition comprising a tetrazolium salt is added to the sample. In one embodiment, the tetrazolium salt is 2-(4-iodophenyl)-3-(4-nitrophenyl)-5-phenyl-2H-tetrazolium chloride (INT)).

When the sampling pad of the sampling device is immersed in the buffered sample, the tetrazolium salt of the coloring composition, the sample, and any other reagents present penetrate into the pad and come into contact with the HDH enzyme. If histamine is present in the sample, the histamine will be oxidatively deamidated by the HDH enzyme. The reduced enzyme will then react with the tetrazolium salt to convert it to a colored product, so the cellulose pad will turn from colorless to colored. For example, if the tetrazolium salt is INT, the sampling pad turns reddish when histamine is present in the sample due to the reaction of the reduced enzyme with INT to form formazan. If histamine is not present in the sample, the sampling pad does not change color. (See FIG. 1) Although the tetrazolium salt can be incubated with the sample, the enzyme is preferred to be impregnated into the sampling pad. If the enzyme is outside the sampling pad, the enzyme can be inhibited by sample matrix components. By impregnating the enzyme into the sampling pad, the sampling pad inhibits interfering substances from entering and binding to the enzyme, while histamine in the sample flows freely into the pad where it can bind to the enzyme.

Unlike other published colorimetric HDH reactions to detect histamine, intermediate electron carriers such as 1-methoxyphenazine methosulfate (mPMS) are not required if INT is used as the tetrazolium salt, since INT is directly oxidized by the reduced enzyme to form a red formazan product, as shown in the following scheme:

Step 1: Histamine+H₂O+HDH_{oxidized}→IAA+NH₃+HDH_{reduced}

Step 2: HDH_{reduced}+INT→HDH_{oxidized}+formazan (colored)

If electron carriers such as mPMS are present, the assay can be performed using other tetrazolium salts such as XTT (2,3-Bis(2-methoxy-4-nitro-5-sulfophenyl)-2H-tetrazolium-5-carboxanilide salt), NBT (nitroblue tetrazolium chloride), MTT (2-(4,5-dimethyl-2-thiazolyl)-3,5-diphenyl-2H-tetrazolium bromide), MTS (5-[3-(carboxymethoxy)phenyl]-3-(4,5-dimethyl-2-thiazolyl)-2-(4-sulfophenyl)-2H-tetrazolium inner salt), WST-1 (sodium 5-(2,4-disulfophenyl)-2-(4-iodophenyl)-3-(4-nitrophenyl)-2H-tetrazolium inner salt), or TTC (2,3,5-triphenyl-2H-tetrazolium chloride). When an electron carrier is used, the electron carrier reacts with the reduced HDH enzyme to form a reduced electron carrier. The reduced electron carrier reacts with the tetrazolium salt altering the color of the tetrazolium salt.

The reactions occur quite rapidly. In most embodiments, the colorimetric sampling test can be performed in less than 5 minutes. The color change of the sampling pad is proportional to the histamine concentration in the sample over a wide concentration range. The amount of histamine present in the sample can be determined by visual comparison of the sampling pad color to that of a control reaction containing a known concentration of histamine or to a known legend of color markers corresponding to specific histamine concentrations printed onto a reference card.

Food and beverage samples can sometimes contain substances which interfere with enzymatic determination of histamine using HDH enzyme. In previously developed liquid assays, samples are treated by heating or solvent extraction to remove interfering substances from the sample. In an embodiment, the sampling pad is designed to block the penetration of interfering substances into the HDH-impregnated sampling pad. Therefore, for many sample types such as seafood and milk, the assay can be efficiently performed even using samples which have not been treated with heat or organic solvents to remove interfering substances. This major simplification of sample preparation provides a significant benefit compared to existing liquid-based histamine dehydrogenase assays for histamine determination.

The amount of histamine in a sample can also be measured with the strips using a lateral flow strip reader which has been calibrated using standards of known histamine concentration.

### EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Production of an Enzymatic Dipstick and Liquid Reagents to Detect Histamine in Liquid Samples

Dissolve 500 units histamine dehydrogenase (from Rhizobium sp. 4-9) into 100 ml 0.2 M glycine pH 9.3. Add 25 ml 10% trehalose and gently mix. Use this enzyme composition to soak CF7 cellulose membrane (GE Whatman) cut into 0.7 cm×30 cm strips which have been attached to the bottom edge (using pressure-sensitive adhesive) of a 30 cm×6 cm inert polymeric card. Dry for 2 hours at 40° C. in a forced air oven. Cut the card into 6 cm×0.45 cm strips. Store the strips in a desiccated container at 4° C.

Prepare a coloring composition by dissolving 60 mg INT in 50 ml deionized water. The neutralization buffer for the sample contains 0.6 M glycine titrated to a pH value of 9.6 using sodium hydroxide. Positive controls can be prepared by diluting a 1000 ppm histamine solution into 0.05% acetic acid solution to a desired histamine concentration for testing. Histamine can be omitted from this solution to produce a negative control (0 ppm histamine).

### Example 2

### Detection of Histamine in Tuna Using an Enzymatic Dipstick.

A 15-30 g portion of a sample of tuna was homogenized in a blender or food homogenizer. Transfer a 4 g portion of the homogenized sample to a clean 50 ml polypropylene screw cap tube. Add 16 ml of 0.05% acetic acid. Mix vigorously for 30 seconds. Incubate one minute. Mix again vigorously for 30 seconds. Allow to stand upright for 5 minutes to allow solid debris to settle. Transfer 0.2 ml of sample (or positive or negative control) to a clean 96 well polystyrene microplate well containing 0.02 mL coloring composition and 0.02 ml neutralization buffer.

Immerse the sampling pad of a sampling device prepared according to Example 1 into the well containing the sample/reagent composition for one minute. Remove the sampling pad from the sample and incubate for 3 minutes at room temperature. Determine the amount of histamine in the sample by comparing the color of the sampling pad to that of a color reference legend or positive or negative control reactions using known amounts of histamine. The color change caused by a 50 ppm spike was very close to the color change observed by the corresponding histamine spike in a buffer composition.

### Example 3

Detection of Histamine in Milk using an Enzymatic Dipstick.

Combine 10 ml milk with 10 ml of 0.05% acetic acid in a plastic 50 ml screw cap tube. Mix gently for 10 seconds. Transfer 0.2 ml of sample (or positive or negative control) to a clean 96 well polystyrene microplate well containing 0.02 ml coloring composition and 0.02 ml neutralization buffer.

Immerse the sampling pad of a sampling device prepared according to Example 1 into the well containing sample/reagent composition for one minute. Remove the sampling pad from the pad and incubate for 3 minutes at room temperature. Determine the amount of histamine in the sample by comparing the color of the cellulose pad to that of a color reference legend or positive or negative control reactions using known amounts of histamine. The observed color change caused by the histamine in milk samples is very close to that observed for control reactions using a buffer containing known amounts of histamine, indicating that the milk sample does not interfere with the enzymatic colorimetric reaction.

### Example 4

### Detection of Histamine in Cheese Using an Enzymatic Dipstick.

Homogenize a 15-30 g portion of sample in a blender or food homogenizer. Transfer a 4 g portion of the homogenized sample to a clean 50 ml polypropylene screw cap tube. Add 16 ml of 0.05% acetic acid. Mix vigorously for 30 seconds. Incubate one minute. Mix again vigorously for 30 seconds. Allow to stand upright for 5 minutes to allow solid debris to settle.

Transfer 0.2 ml of sample (or positive or negative control) to a clean 96 well polystyrene microplate well containing 0.02 ml coloring composition and 0.02 ml neutralization buffer.

Immerse the sampling pad of a sampling device prepared according to Example 1 into the well containing sample/reagent composition for one minute. Remove the sampling pad from the pad and incubate for 3 minutes at room temperature. Determine the amount of histamine in the sample by comparing the color of the cellulose pad to that of a color reference legend or positive or negative control reactions using known amounts of histamine. The observed color change caused by the histamine in cheese samples is very close to that observed for control reactions using buffer containing known amounts of histamine, indicating that the cheddar cheese sample does not interfere with the enzymatic colorimetric reaction.

Further modifications and alternative embodiments of various aspects of the invention will be apparent to those skilled in the art in view of this description. Accordingly, this description is to be construed as illustrative only and is for the purpose of teaching those skilled in the art the general manner of carrying out the invention. It is to be understood that the forms of the invention shown and described herein are to be taken as examples of embodiments. Elements and materials may be substituted for those illustrated and described herein, parts and processes may be reversed, and certain features of the invention may be utilized independently, all as would be apparent to one skilled in the art after having the benefit of this description of the invention. Changes may be made in the elements described herein without departing from the spirit and scope of the invention as described in the following claims.

What is claimed is:

1. A method of detecting histamine in a sample, comprising:
obtaining a liquid sample containing histamine from the sample, wherein the liquid sample is buffered at a pH of between about 8.5 to about 10;
adding a coloring composition comprising a tetrazolium salt to the liquid sample to create a test sample;
placing a sampling device into the test sample, the sampling device comprising:
a sampling pad impregnated with histamine dehydrogenase enzyme; and
a support substrate, wherein the sampling pad is coupled to the support substrate;
wherein the pad of the sampling device is placed into the test sample;
removing the sampling device from the test sample;
allowing the sampling pad to incubate for a time of less than five minutes, wherein a change in color of the sampling pad indicates the presence of histamine in the sample.

2. The method of claim 1, wherein preparation of the liquid sample comprises:
treating a solid sample with an aqueous acidic composition to form an acidic mixture;
removing undissolved solid from the acidic mixture to form a clarified acidic mixture; and
adding at least a portion of the clarified acidic mixture to a neutralization buffer to form the liquid sample.

3. The method of claim 1, wherein preparation of the liquid sample comprises:
treating a liquid sample with an aqueous acidic composition to form an acidic mixture;
adding at least a portion of the clarified acidic mixture to a neutralization buffer to form the liquid sample.

4. The method of claim 1, wherein the tetrazolium salt is 2-(4-iodophenyl)-3-(4-nitrophenyl)-5-phenyl-2H-tetrazolium chloride.

5. The method of claim 1, wherein the coloring composition further comprises an electron carrier, wherein the electron carrier reacts with reduced HDH enzyme to form a reduced electron carrier, and wherein the reduced electron carrier reacts with the tetrazolium salt altering the color of the tetrazolium salt.

6. The method of claim 1, where the histamine dehydrogenase enzyme is obtained from Rhizobium sp. 4-9.

7. The method of claim 1, wherein the sampling pad is an absorbent pad.

8. The method of claim 1, wherein the sampling pad is a cellulose fiber pad.

9. The method of claim 1, wherein the sampling pad inhibits the penetration of HDH interfering substances into the sampling pad.

10. A sampling device for histamine detection comprising:
a sampling pad impregnated with histamine dehydrogenase enzyme; and
a support substrate, wherein the sampling pad is coupled to the support substrate.

11. The sampling device of claim 10, where the histamine dehydrogenase enzyme is obtained from Rhizobium sp. 4-9.

12. The sampling device of claim 10, wherein the sampling pad is an absorbent pad.

13. The sampling device of claim 10, wherein the sampling pad is a cellulose fiber pad.

14. The sampling device of claim 10, wherein the sampling pad inhibits the penetration of HDH interfering substances into the sampling pad.

15. A test kit for the visual detection of histamine in a sample comprising:
a sampling device comprising:
a sampling pad impregnated with histamine dehydrogenase enzyme; and
a support substrate, wherein the sampling pad is coupled to the support substrate;
a coloring composition comprising a tetrazolium salt; and
a buffer composition having a pH of between about 8.5 to about 10.

16. The test kit of claim 15, where the buffer composition is a glycine buffer.

17. The test kit of claim 15, wherein the coloring composition has a concentration of tetrazolium salt of between about 0.5-1.5 mg/ml.

18. The test kit of claim 15, wherein the coloring composition further comprises an electron carrier.
